(19) United States
(12) Patent Application Publication
(10) Pub. No.: US 2024/0398502 A1
(43) Pub. Date: Dec. 5, 2024

(54) ROBOTIC THREE-DIMENSIONAL DISPLAY MOUNT FOR DIGITAL VISUALIZATION OF OPHTHALMIC SURGERY

(71) Applicant: Alcon Inc., Fribourg (CH)

(72) Inventors: Patrick TERRY, Goleta, CA (US); Sumit PALIWAL, Irvine, CA (US); Mark Andrew ZIELKE, Lake Forest, CA (US); Lance NOLLER, Yorba Linda, CA (US)

(21) Appl. No.: 18/677,596

(22) Filed: May 29, 2024

Related U.S. Application Data
(60) Provisional application No. 63/505,050, filed on May 30, 2023.

(51) Int. Cl. A61B 90/00; A61B 34/00; A61B 90/20; A61F 9/007

(52) U.S. Cl. CPC A61B 90/37; A61B 34/70; A61B 90/20; A61F 9/007; A61B 2090/368; A61B 2090/372

(57) ABSTRACT

A system includes a display device and an actuated structure mounted to the display device and configured to move the display device in three-dimensional space. The system includes an imaging device configured to capture images of a patient's eye during an ophthalmic surgery and display the images on the display device. A controller is configured to activate the actuated structure to maintain visibility of the display device to a surgeon performing the ophthalmic surgery. The display may be positioned based on a surgeon profile and/or a treatment plan. Whether the display is visible may be determined based on images from a camera mounted to the imaging device, the display device, or elsewhere. Whether the display is visible may be determined based on kinematic data for the actuated structure and/or a support for the imaging device.

### CROSS-REFERENCE TO RELATED APPLICATIONS

This application claim benefit of and priority to U.S. Provisional Patent Application No. 63/505,050, filed May 30, 2023, which is hereby assigned to the assignee hereof and hereby expressly incorporated by reference in its entirety as if fully set forth below and for all applicable purposes.

### INTRODUCTION

Ophthalmic surgery is performed to treat cataracts, glaucoma, and diseases of the retina. During ophthalmic surgery, a surgeon typically looks into the eye pieces of a binocular surgical microscope to view the intricate anatomy of the patient's eye and surgical instruments. However, many hours spent hunched over a surgical microscope leaves many surgeons with cervical injuries.

It would be an advancement in the art to improve the ergonomics of ophthalmic surgery to prevent such injuries.

### SUMMARY

In certain embodiments, a system is provided. The system includes a display device and an actuated structure mounted to the display device and configured to move the display device in three-dimensional space. The system includes an imaging device configured to capture images of a patient's eye during an ophthalmic surgery and display the images on the display device. A controller is configured to activate the actuated structure to maintain visibility of the display device to a surgeon performing the ophthalmic surgery.

In certain embodiments, a system is provided. The system includes a display device and an actuated structure mounted to the display device and configured to move the display device in three-dimensional space. The system further includes an imaging device configured to capture surgical images of a patient's eye during an ophthalmic surgery and display the surgical images on the display device, and a camera mounted to the imaging device. A controller is configured to receive camera images from the camera, evaluate representations of the display device in the camera images, and activate the actuated structure to maintain the display device unobstructed in a field of view of a surgeon performing the ophthalmic surgery based on the representations of the display device.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates an example operating environment including a robotic three dimensional display mount in accordance with certain embodiments.

FIG. 2 is side view of the example operating environment of FIG. 1 showing alternative mounting options for the robotic three-dimensional display mount in accordance with certain embodiments.

FIG. 3 is top view of the example operating environment of FIG. 1 showing various positions for a three-dimensional display in accordance with certain embodiments.

FIG. 4 is a schematic block diagram of inputs for controlling the robotic three-dimensional display mount in accordance with certain embodiments.

FIG. 5 is a process flow diagram of a method for controlling the robotic three-dimensional display mount in accordance with certain embodiments.

FIG. 6 is a diagram illustrating image processing for controlling positioning of the robotic three-dimensional display mount in accordance with certain embodiments.

FIG. 7 illustrates an example computing device that implements, at least partly, one or more functionalities for controlling positioning of the robotic three-dimensional display mount in accordance with certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

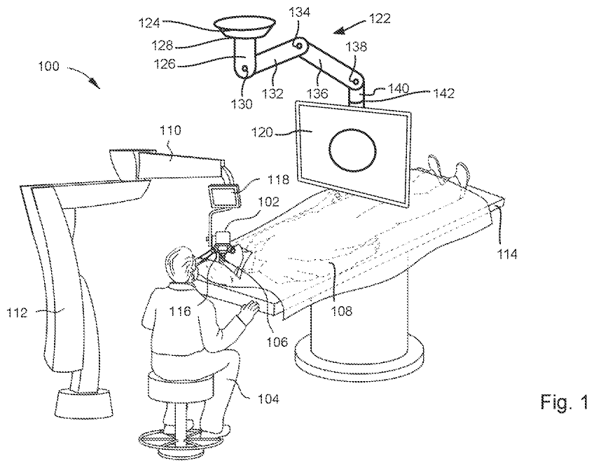

FIG. 1 illustrates an example system 100 in which a robotic display mount may be used. The system 100 includes an ophthalmic microscope 102. A surgeon 104 uses the ophthalmic microscope 102 to visualize structures on and in an eye 106 of a medical patient 108 undergoing a surgery or examination. The microscope 102 is supported on, in this illustration, an adjustable overhead arm 110 of a microscope support pedestal 112. The patient 108 may be supported on an operating table 114. The ophthalmic microscope 102 is movable with the overhead arm 110 in three translational dimensions and possibly one or more rotational directions so that the surgeon 104 can position the ophthalmic microscope 102 as desired with respect to the eye 106 of the patient 108.

In certain embodiments, the ophthalmic microscope 102 comprises a high resolution, high contrast stereo viewing surgical microscope. The ophthalmic microscope 102 will often include a monocular eyepiece 116 or binocular eyepieces 116, through which the surgeon 104 will have an optically magnified view of the relevant eye structures that the surgeon 104 will need to see to accomplish a given surgery or diagnose an eye condition of the patient 108.

The ophthalmic microscope 102 includes a digital camera and broadband light source for capturing color (red, green, and blue) images, a multi-spectral imaging (MSI) device, and/or other type of imaging device. Digital images captured using the camera may be displayed on a display device within the ophthalmic microscope 102.

The ophthalmic microscope 102 may include two display devices viewable through binocular eyepieces 116 and that display images of the patient's eye 106 that are captured from different viewpoints by two cameras to provide stereoscopic viewing. For example, the ophthalmic microscope 102 may be implemented as the NGENUITY 3D VISUALIZATION SYSTEM provided by Alcon Inc. of Fort Worth Texas.

Images from the ophthalmic microscope 102 may be additionally or alternatively be displayed on one or more display devices. For example, the one or more display devices may include a display device 118 fastened to the supporting arm 110 above the ophthalmic microscope 102.

In order to relieve the surgeon from the need to constantly look into the eye pieces 116 to obtain a stereoscopic view, a display device 120 may be implemented as a three-dimensional display device. The display device 120 may therefore provide a stereoscopic view of images captured using the ophthalmic microscope 102. The display device 120 may be embodied as any type of three-dimensional display device known in the art, including those that do or do not use special filtering glasses. For some types of three-dimensional display devices, the perception of three dimensions requires that the distance of the viewer from the display device 120 be within a threshold distance from the display device.

The display device 120 may advantageously be large to enable viewing of important details of the anatomy of the patient's eye 106 and surgical instruments. For example, the display device 120 may have a size greater than a 35-inch diagonal, 45 inch diagonal, 55-inch diagonal, 65 inch diagonal, or larger. The display device 120 may have a resolution of at least 1080p, 1440p, 2160p, 2540p, 4000p, or 4320p, as defined by the Advanced Television Systems Committee (ATSC) or some other custom resolution. The display device 120 is preferably a color display device 120 and may be implemented as a light emitting diode (LED) display, liquid crystal display (LCD), organic LED (OLED) display, digital light projection (DLP), or any other type of display technology.

Although a display device 120 can help improve the posture of the surgeon to help avoid cervical strain and injury, positioning of the display for each procedure takes a few minutes. Likewise, movement of the surgeon 104, the ophthalmic microscope 102, or other personnel and equipment may require repositioning of the display device 120 during a surgery. In some cases, if the display device 120 becomes obstructed, the surgeon 104 may return to using the eye pieces 116 of the ophthalmic microscope 102, thereby negating the benefit of the display device 120.

The display device 120 is mounted to a robotic arm 122 that is controlled to maintain the display device 120 visible to the surgeon at the start of the surgery and adjusts the position of the display device 120 during surgery to maintain visibility of the display device 120. As used herein “maintain visibility” may be understood as including at least maintaining a line of sight to the entirety of the display device and may also be understood as maintaining a distance and orientation of the display device 120 relative to the surgeon's eyes that one or both of (a) are within a tolerance of a preferred distance and orientation of the surgeon or other predefined acceptable range of distances and orientations and (b) enable three-dimensional perception of images displayed on the display device 120 by the surgeon, such as may be defined by a manufacturer of the display device 120.

The robotic arm 122 may have four, five, or six degrees of freedom (DOF). For example, the robotic arm 122 may be embodied as a six DOF serial robotic arm. The robotic arm 122 may be mounted to a base 124 mounted to a ceiling or elsewhere in the operating room including the system 100. The robotic arm 122 may also be mounted to a mobile cart that may be positioned adjacent to the operating table 114. A first link 126 is mounted to the base 124 by means of a shoulder joint 128. For example, the joint 128 may be a rotational joint enabling the joint 128 to spin between 45 and 360 degrees. A rotary junction may be used to couple signals to actuators of the robotic arm 122 and the display device 120 such that rotation of more than 360 degree may be performed.

The link 126 may be connected by joint 130 to a link 132. The link 132 may be connected by joint 134 to a link 136 and the link 136 may be connected by joint 138 to a link 140. The joints 130, 134, 138 are embodied as elbow joints in the illustrated embodiment. The link 140 may be coupled by joint 142 to a mounting point to the display device 120. The joint 142 may be a rotational joint enabling the display device 120 to rotate about an axis relative to the link 140.

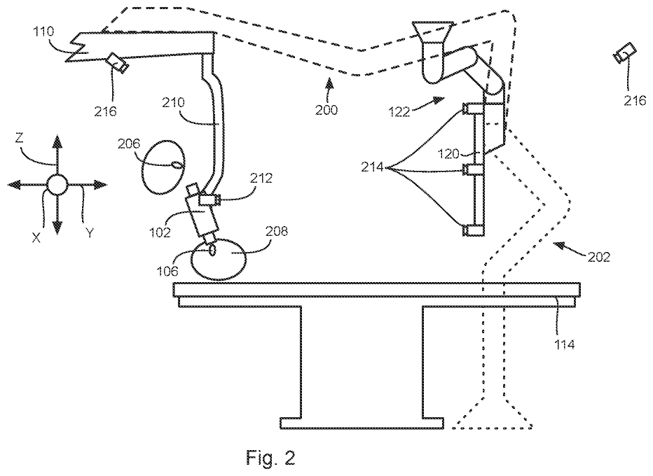

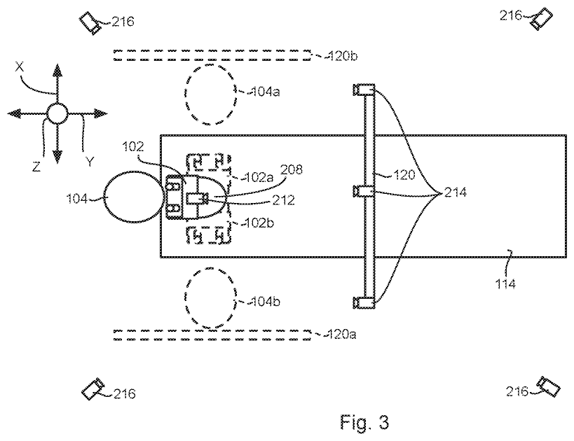

In the illustrated example, five joints 128, 130, 134, 138, 142 are shown enabling five DOF, which is sufficient to enable the display device 120 to be positioned in view of the surgeon 104 for typical ophthalmic procedures. Where the robotic arm 122 is a commercially available robotic arm, six DOF may be available with the sixth DOF being unused. Alternatively, all six DOF may be used to provide more flexibility in determining kinematic solutions for positioning the display device 120 at a desired position and orientation, to reduce footprint, increase the range of reachable positions, or otherwise improve performance of the robotic arm 122. For example, a sixth DOF may enable the display device 120 to rotate about a line normal to the screen of the display device 120, which is not needed for most applications. However, in other embodiments, such rotation is used, such as to change the orientation of the wider dimension of the display between vertical and horizontal orientations. In other embodiments, the sixth DOF may be used to change tilt of the display device 120 in a plane parallel to the Z direction (see definition of Z direction in FIGS. 2 and 3).

The illustrated robotic arm 122 is one example of an actuated structure that may be used to control the position and orientation of the display device 120. Other actuated structures, such as other types of robotic arms, gantries, or other collections of actuators and links may also be used, including those including translational joints in place of or in addition to rotational joints.

Referring to FIG. 2, the robotic arm 122 may be mounted to the overhead arm 110 in the illustrated position 200 or mounted to a floor in the illustrated position 202. The robotic arm 122 may also be mounted to a wall, the pedestal 112, the operating table 114, or on a mobile cart that may be rolled relative to the floor.

As disclosed in greater detail below, the robotic arm 122 is automatically controlled to maintain the entire screen of the display device 120 viewable by both eyes 206 of the surgeon 104. The robotic arm 122 may therefore be actuated to move the display device 120 to a position that is not obstructed by the ophthalmic microscope 102 or other obstruction and is facing the surgeon's current position.

The position of the surgeon's eyes 206 and the display device 120 may be defined with respect to an X (horizontal), a Y (longitudinal), and a Z (vertical, i.e., the direction of gravity) direction that are mutually orthogonal. The operating table 114 may be adjusted in the Z direction to account for the height of patient's head 208 such that the patient's eye 106 is at a desired height to be operated on by the surgeon 104. The position of the display device 120 in the Z direction may be automatically controlled such that the ophthalmic microscope 102 is not intercepted by a ray extending between the surgeon's eyes 206 and any point on the screen of the display device 120. The position of the display device 120 in the X-Y plane may be selected to avoid other potential obstructions, such as a link 210 suspending the ophthalmic microscope from the overhead arm 110, other personnel present in the operating room, other equipment, or the like.

Automatic positioning of the robotic arm 122 may be facilitated by means of one or more cameras. In one embodiment, one or more cameras 212 are mounted to the ophthalmic microscope 102 and has the field of view thereof pointing away from the surgeon 104. The screen of the display device 120 is in the field of view of the camera 212 and the output of the camera 212 is used to infer the relative location of the ophthalmic microscope 102 and the display device 120. The relative location may then be used to adjust the position of the display device 120 using the robotic arm 122 to place a greater extent of the display device 120 in the field of view of the surgeon's eyes.

In other embodiments, cameras 214 are mounted to the display device 120, such as around a perimeter of the screen of the display device 120. The outputs of the cameras 214 may be evaluated to determine whether the surgeon's eyes 206 are in the field of view of each camera 214. The position of the display device 120 may be adjusted using the robotic arm 122 to ensure that the surgeon's eyes 206 are in the field of view of all of the cameras 214.

In still other embodiments, one or more cameras 216 are mounted at one or more locations in an operating room, such as on the overhead arm 110, pedestal 112, one or more walls, or the ceiling. The outputs of the cameras 216 may be used to determine the location of the surgeon's eyes 206, the orientation of the surgeon's head, the position and orientation of the ophthalmic microscope 102, the position of other potential obstructions, and/or the position and orientation of the display device 120. The position and/or orientation information obtained using the cameras 216 may then be used to adjust the position and orientation of the display, if necessary, to place a greater extent of the display device 120 in the field of view of the surgeon's eyes.

Referring to FIG. 3, the display device 120 may be placed in various positions based on the procedure being performed by the surgeon 104. For example, when performing retinal surgery, the surgeon 104 may sit in the illustrated superior position. When performing cataract surgery, the surgeon 104 may sit in the left temporal position 104a with the ophthalmic microscope 102 in position 102a to operate on the left eye and sit in the right temporal position 104b with the ophthalmic microscope 102 in position 102b to operate on the right eye.

The display device 120 is automatically positioned by the robotic arm 122 based on the procedure being performed without requiring the surgeon or other operator to do so and avoiding any delay required for manual positioning and repositioning. For example, the display device 120 may be in the illustrated position when the surgeon 104 is at the superior position. The display may be in the position 120a when the surgeon is in the left temporal position and in the position 120b when the surgeon is in the right temporal position. As is apparent, in the illustrated position, the screen of the display device 120 is substantially (e.g., within 5 degrees of) parallel to the X-Z plane. In the positions 120a, 120b, the screen of the display is substantially parallel to the Y-Z plane. In use, the display device 120 may be moved by the robotic arm 122 within a range of positions about those illustrated to avoid obstructions.

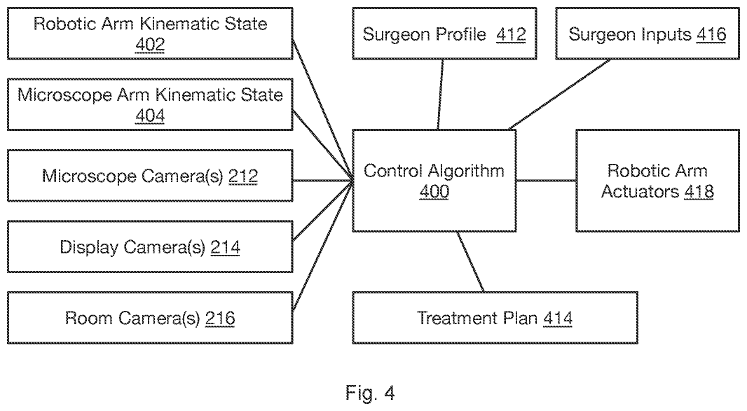

FIG. 4 illustrates components and data that may be used to control the robotic arm 122 in order to maintain visibility of the display device 120 to the eyes 206 of the surgeon 104. A control algorithm 400 may be executed by a controller embodied as a distinct computing device or a computing device incorporated into one or more of the ophthalmic microscope 102, the robotic arm 122, and the display device 120.

The control algorithm 400 may receive, as an input, a kinematic state 402 of the robotic arm 122. As used herein, “kinematic state” may be understood as a relative orientation of links of a robotic arm and possibly the velocity and acceleration of the links of the robotic arm. A kinematic state may include, or be sufficient to derive, a position and orientation in three-dimensional space of an end of the robotic arm. For example, the kinematic state 402 may include the relative positions and orientations of the links 126, 132, 136, 140 of the robotic arm 122 relative to one another, the base 124, and the display device 120. The kinematic state 402 may further include the velocity and acceleration of the links 126, 132, 136, 140. The kinematic state 402 may include, or be sufficient to derive, a position and orientation in three-dimensional space of the display device 120.

The control algorithm 400 may receive, as an input, a kinematic state 404 of an a support for the ophthalmic microscope 102, such as some or all of the pedestal 112, overhead arm 110, the link 210 suspending the ophthalmic microscope from the overhead arm 110, joints between these members and any other links or joints supporting the ophthalmic microscope 102. The kinematic state 404 may include the relative positions and orientations of the pedestal 112, overhead arm 110, link 210, or one or more other links and joints. The kinematic state 404 may further include the velocity and acceleration of some or all of the pedestal 112, overhead arm 110, link 210, or one or more other links and joints. The kinematic state 404 may include, or be sufficient to derive, a position and orientation in three-dimensional space of the ophthalmic microscope 102.

In embodiments where the kinematic states 402, 404 are available and provide the location and orientation in three-dimensional space of the ophthalmic microscope 102 and the display device 120, whether or not the display device 120 is obstructed may be determined exclusively from the states 402, 404. For example whether the display device 120 is obstructed may be determined from inputs including some or all of the known positions and orientations of the ophthalmic microscope 102 and the display device 120, an assumed position of the surgeon's eyes 206 relative to the ophthalmic microscope 102, and possibly other information from the treatment plan 414 such as some or all of a microscope working distance relative to the eye 106 of the patient, patient bed height, and patient head height. Using these inputs, whether the surgeon's eyes 206 are obstructed by the ophthalmic microscope 102, a link 210, or other link supporting the ophthalmic microscope 102 may be determined and a new position for the display device 120 may be calculated (e.g., using a kinematic reachability calculation) and the robotic arm 122 commanded to achieve the new position.

In embodiments where the kinematic states 402, 404 are not available, or where more accurate assessments of the visibility of the display device 120 are needed or desired, the control algorithm 400 may further take, as inputs, images output by some or all of one or more cameras 212 mounted to the ophthalmic microscope 102, one or more cameras 214 mounted to the display device 120, and one or more other cameras 216 mounted in the operating room, overhead arm 110, pedestal 112, or elsewhere. The control algorithm 400 receives images from some or all of the cameras 212, 214, 216, estimates the relative position of the display device 120 and the surgeon's eyes 206, estimates obstruction of the display, and determines an appropriate adjustment of the position and orientation of the display device 120 in order to remove the obstruction. For example, the surgeon may be in a hunched or reclined position or some intermediate position or at any position in between at any point in a surgery, which will affect the position (particularly height) and gaze direction of the surgeon's eyes. Tracking of the position and gaze direction of the surgeon's eyes 206 using images from the one or more cameras 216 may therefore be used to maintain visibility of the display device 120 as the surgeon changes position.

The control algorithm 400 further takes, as inputs, data to facilitate determining an initial position for the display device 120. For example, the control algorithm 400 may receive one or both of a surgeon profile 412 and a treatment plan 414. The surgeon profile 412 corresponds to a surgeon 104 performing a procedure using the ophthalmic microscope 102 and display device 120. The surgeon profile 412 may include a height of the surgeon 104, a height of the surgeon's eyes 206 when seated and performing a procedure, a preferred position of the ophthalmic microscope relative to the surgeon's eyes 206, or other information. The surgeon profile 412 may include measurements of the position of the eyes 206 of the surgeon relative to the ophthalmic microscope in general or for a particular procedure. The surgeon profile 412 may include a preferred viewing angle of the surgeon's eyes 206. The measurements of the surgeon profile 412 may be obtained manually or determined from images obtained using the cameras 214, 216 during a prior procedure.

The treatment plan 414 defines steps of a procedure to be performed. The treatment plan 414 may include guidance to the surgeon in terms of text, overlays superimposed on images captured using the ophthalmic microscope 102, or other information. The treatment plan 414 may indicate the type of procedure and the eye being operated on, e.g., retinal surgery, cataract surgery, glaucoma surgery. The position of the surgeon (superior, temporal left, temporal right) may be inferred from the treatment plan 414 or the treatment plan 414 may indicate the position of the surgeon. In some instances, the treatment plan 414 may require the surgeon to change position, e.g., from superior to temporal left. This change may be specified explicitly or may be determinable by treatments specified in the treatment plan.

The control algorithm 400 receives the treatment plan 414 and determines the position of the display device 120 at the start of the surgery corresponding to the surgeon position (see, e.g., FIG. 3 and corresponding discussion). The control algorithm 400 may interface with the ophthalmic microscope, which presents guidance according to the treatment plan 414, accordingly, upon reaching a stage in the treatment plan requiring a change in surgeon position to a new position, the control algorithm 400 may detect the change and adjust the position and orientation of the display device 120 to correspond to the new surgeon position.

The control algorithm 400 may further receive surgeon inputs 416. The surgeon inputs 416 may include buttons, a touch screen, voice controls, gesture controls, or other interfaces for receiving inputs from the surgeon 104. A surgeon may use the inputs 416 to directly command changing of the display device 120 to a position corresponding to superior, temporal left, or temporal right positions. The surgeon or other operator may also use the inputs 416 to command the robotic arm 122 to adjust the position and orientation of the display device 120, e.g., up down, left, right, or rotation in the X-Y plane or a plane parallel to the Z direction. The surgeon may use the inputs 416 to suppress automatic adjustment of the position and orientation of the display device 120 or to invoke automatic adjustment of the position and orientation of the display device 120. In some embodiments, the robotic arm 122 includes force and/or torque sensors such that force exerted on the display 120 or robotic arm 122 by the surgeon or other operator may be detected and used as an input to invoke changing of the position and/or orientation of the display 120.

The control algorithm 400 produces an output transmitted to the actuators 418 of the joints of the robotic arm 122, such as the joints 128, 130, 134, 138, 142 in the example of FIG. 1 or other joints for other types of robotic arms. The output of the control algorithm 400 may be in the form of a desired position and orientation of the display device 120 and a separate algorithm may determine changes to the angles of the joints in order to achieve the position and orientation, such as according to a kinematic model of the robotic arm 122 and sensors providing the current kinematic state 402 of the robotic arm 122.

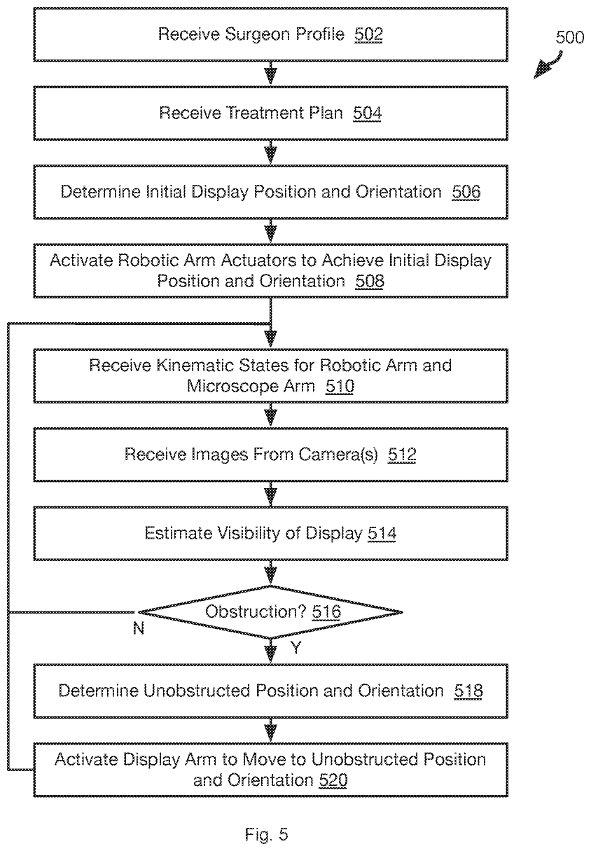

Referring to FIG. 5, the control algorithm 400 may execute the illustrated method 500 with respect to an ophthalmic microscope 102, surgeon 104, display device 120, and robotic arm 122, and possibly one or more cameras 212, 214, 216.

The method 500 includes receiving, at step 502, the surgeon profile 412 and receiving, at step 504, the treatment plan 414 and determining, at step 506, an initial position and orientation for the display device 120 relative to the ophthalmic microscope 102. For example, the surgeon profile 412 indicates a height of the surgeon's eyes 206 and preferred height of the ophthalmic microscope 102. The treatment plan 414 indicates the surgeon position (superior, temporal left, temporal right). The initial position is therefore determined as a position and orientation in the X-Y plane facing the surgeon position (see FIG. 3 and corresponding description) and a height in the Z direction selected such that the screen of the display device 120 is visible for the given height of the surgeon's eyes, the height of the microscope 102, and possibly based on positions of the link 210 or other known structures. The initial position may include selecting a tilt in a plane parallel to the Z direction and an angle in the X-Y plane such that a normal vector of the screen is pointed approximately (e.g., within 5 degrees) at the expected location of the surgeon's eyes. For example, the expected location of the surgeon's eyes 206 may be at the height of the surgeon's eyes in the Z direction as indicated in the surgeon profile 412 and at a predefined offset from the ophthalmic microscope 102 in a plane intersecting the center of the ophthalmic microscope 102, e.g., a plane passing between the eye pieces 116. The predefined point may be a preferred position of the surgeon relative to the ophthalmic microscope 102 as indicated in the surgeon profile 412. Where the display device 120 is a three-dimensional display device, the initial position may be selected to be within a threshold distance and within a threshold angle relative to the surgeon's eyes 206 relative to an ideal orientation such that three-dimensional perception is possible.

The method 500 includes activating, at step 508, one or more actuators of the robotic arm 122 in order to achieve the initial position and orientation of the display device 120 as determined at step 506. Note that where a treatment plan 414 specifies a change in surgeon position to a new position during a procedure, steps 506 and 508 may be repeated for the new position upon reaching the point in the procedure where the change in position is specified.

In some embodiments including the use of cameras 214, 216 providing images enabling the relative location of the surgeon 104 and ophthalmic microscope 102 to be determined, steps 502-508 may be omitted. For example, the surgeon may sit in a desired position and arrange the ophthalmic microscope 102 in a desired position. The control algorithm may determine the position of the surgeon's eyes 206 and ophthalmic microscope 102, determine a location for the display device 120 that is unobstructed by the ophthalmic microscope 102, link 210, or other structures and facing the surgeon's eyes 206, e.g., a normal vector of the screen of the display device 120 pointing, within 5 degrees, at the surgeon's eyes 206.

During a procedure, movement of the surgeon 104, ophthalmic microscope 102, and other equipment or personnel that obstructs the surgeon's view of the display device 120 may be detected and compensated for with movement of the display device 120. For example, the method 500 may include receiving, at step 510, the kinematic state 402, 404 for one or both of the robotic arm 122 and an arm supporting the ophthalmic microscope 102. The method 500 may include receiving, at step 512, images from some or all of the cameras 212, 214, 216. Note that in some embodiments, only one of steps 510 and 512 is performed.

The method 500 includes estimating, at step 514, a visibility of the display device 120 based on one or both of (a) the kinematic state 402, 404 and (b) images from some or all of the cameras 212, 214, 216.

The kinematic state 402, 404 may be used to estimate visibility of the display device 120 by using known locations of the ophthalmic microscope 102 and display device 120 and inferred location (e.g., from surgeon profile) of the surgeon's eyes 206 to determine whether the ophthalmic microscope 102 is in the line of sight of the surgeon's eyes 206.

When using cameras 216, determining visibility may be determined similarly to the approach used for kinematic state 402, 404. Images from the cameras 216 may be evaluated to determine the locations of the ophthalmic microscope 102, display device 120, the surgeon's eyes 206, and any obstructions. Using this information, whether the entire display device 120 is in the field of view of the surgeon's eyes and unobstructed may be determined, such as using ray tracing between the surgeon's eyes and a collection of points distributed over the display device 120.

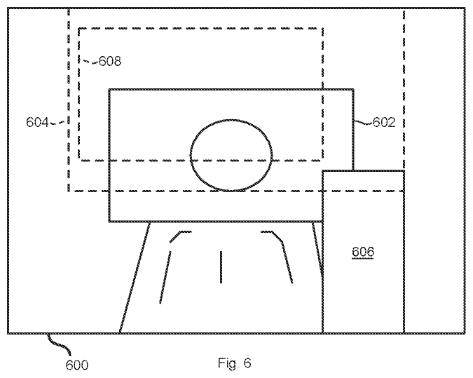

Referring to FIG. 6, while still referring to FIG. 5, step 514 may include using images from some or all of the cameras 212, 214, 216 to estimate visibility of the display device 120. For example, an image 600 captured with the camera 212 includes a representation 602 of the display device 120. The representation of the display device 120 may be recognized by identifying the image captured by the ophthalmic microscope 102 displayed on the display device 120.

The location of the representation 602 in the image may be compared to a predefined region 604 corresponding to locations in which the display device 120 is likely to be visible to the surgeon's eyes 206. The region 604 may be general to all users or may take into account the surgeon profile 412, e.g., the height of the surgeon's eyes 206 indicated in the surgeon profile. If the representation 602 of the display device 120 is not located completely within the region, the display device 120 may be determined to be obstructed.

The image 600 may further be analyzed to see if any portion of the image received from the ophthalmic microscope 102 is not visible in the image 600. For example, the image 600 includes the illustrated representation 606 of an obstruction to visibility of the display device 120 and this representation 606 may be identified at step 514.

Using images from the cameras 214, step 514 may include evaluating whether the surgeon's eyes 206 are visible in each image from each camera 214. If the surgeon's eyes are not visible in an image from at least one camera 214, the display device 120 may be determined to be obscured.

If the display device 120 is found, at step 516, to be obstructed, the method 500 may include determining, at step 518, an unobstructed location. For example, continuing with the example of FIG. 6, step 518 may include determining that location 608 is within the region 604 and also does not overlap the obstruction represented by the representation 606. For example, step 518 may include moving the display device 120 in the opposite direction relative to a portion of the representation 602 that is obstructed or outside the region 604. For example, in FIG. 6, the display device 120 may be moved up in response to the representation 602 extending below the region 604. The display device 120 may be moved to the left in response to the right side of the representation 602 being obscured.

Using images from the cameras 214, step 518 may include moving the display device 120 in the opposite direction from a camera 216 for which a received image does not have the eyes 206 of the surgeon 104 in the field of view thereof. For example, if a camera 216 on a left edge of the display device 120 does not have the eyes 206 of the surgeon in the field of view thereof, the display device 120 may be moved to the right. If a camera 216 on the bottom edge of the display device 120 does not have the eyes 206 of the surgeon 104 in the field of view thereof, the display device 120 may be moved upward.

Using the kinematic state 402, 404 and/or locations determined using images from the cameras 216, step 518 may include determining a new location for the display device 120 in which the estimated visibility of the display device 120 is improved relative to the current estimated visibility based on a known location of the ophthalmic microscope 102 and an inferred or detected location of the eyes 206 of the surgeon 104. In some embodiments, both the kinematic states 402, 404 and images from the cameras 216 are used. For example, images from the cameras 216 may be used to determine the location of obstructions other than the ophthalmic microscope 102. Step 518 may therefore include identifying a position and orientation of the display device 120 that is visible based on the known location of the ophthalmic microscope 102 and any identified obstructions.

In any of the above example implementations of step 518, the determined position and orientation of the display device 120 may position the display device 120 within the threshold distance from the inferred or detected location of the surgeon's eyes 206 in order to enable stereoscopic vision where the display device 120 is a three-dimensional display device.

The method 500 includes activating, at step 520, actuators of the robotic arm 122 to move the display device 120 to the unobstructed location determined at step 518. Processing may continue at step 510 in order to adapt to subsequent obstructions of the display device 120 and to verify that the movement determined at step 518 successfully removed obstruction of the display device 120.

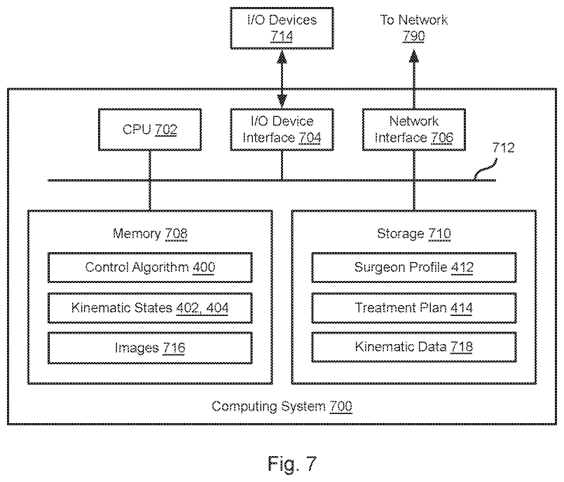

FIG. 7 illustrates an example computing system 700 that implements the controller executing the control algorithm 400. The ophthalmic microscope 102 and the display device 120 may likewise incorporate a computing device having some or all of the attributes of the computing system 700.

As shown, computing system 700 includes a central processing unit (CPU) 702 (and possibly a graphics processing unit (GPU)), one or more I/O device interfaces 704, which may allow for the connection of various I/O devices 714 (e.g., keyboards, displays, mouse devices, pen input, etc.) to computing system 700, network interface 706 through which computing system 700 is connected to network 790, a memory 708, storage 710, and an interconnect 712.

CPU 702 may retrieve and execute programming instructions stored in the memory 708. Similarly, CPU 702 may retrieve and store application data residing in the memory 708. The interconnect 712 transmits programming instructions and application data, among CPU 702, I/O device interface 704, network interface 706, memory 708, and storage 710. CPU 702 is included to be representative of a single CPU, multiple CPUs, a single CPU having multiple processing cores, and the like.

Memory 708 is representative of a volatile memory, such as a random access memory, and/or a nonvolatile memory, such as nonvolatile random access memory, phase change random access memory, or the like. As shown, memory 708 may store executable code implementing the control algorithm 400 and data used by the control algorithm 400 such as kinematic states 402, 404 and images 716 from some or all of the cameras 212, 214, 216.

Storage 710 may be non-volatile memory, such as a disk drive, solid state drive, or a collection of storage devices distributed across multiple storage systems. Storage 710 may optionally store information such as the surgeon profile 412, treatment plan 414, and kinematic data 718 describing the robotic arm 122 and possibly an arm supporting the ophthalmic microscope 102 to enable points in three-dimensional space to be related to states of joints of the robotic arm and possibly the arm supporting the ophthalmic microscope 102.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A system comprising:
a display device;
an actuated structure mounted to the display device and configured to move the display device in three-dimensional space;
an imaging device configured to capture images of a patient's eye during an ophthalmic surgery and display the images on the display device; and
a controller configured to activate the actuated structure to maintain the display device in a field of view of a surgeon performing the ophthalmic surgery.

2. The system of claim 1, wherein the imaging device is an ophthalmic microscope.

3. The system of claim 1, wherein the imaging device is a stereoscopic ophthalmic microscope.

4. The system of claim 3, wherein the display device is a three-dimensional display device and the controller is configured to activate the actuated structure to maintain an orientation and distance of the display device relative to the surgeon to enable three-dimensional perception by the surgeon.

5. The system of claim 1, wherein the controller is configured to activate the actuated structure to maintain the display device in the field of view of the surgeon performing the ophthalmic surgery by:
receiving a surgeon profile;
selecting a position and orientation of the display device according to the surgeon profile; and
activating the actuated structure to achieve the position and orientation.

6. The system of claim 1, wherein the controller is configured to activate the actuated structure to maintain the display device in the field of view of the surgeon performing the ophthalmic surgery by:
receiving a treatment plan for the ophthalmic surgery;
selecting a position and orientation of the display device according to the treatment plan; and
activating the actuated structure to achieve the position and orientation.

7. The system of claim 6, wherein the treatment plan specifies a surgeon position selected from superior, left temporal, and right temporal; and
wherein the controller is further configured to select the position and orientation of the display device such that the display device is visible from the surgeon position in the treatment plan.

8. The system of claim 7, wherein the controller is further configured to select a height of the display device according to a surgeon profile of the surgeon.

9. The system of claim 1, wherein the actuated structure is a robotic arm having at least 5 degrees of freedom.

10. The system of claim 1, wherein the controller is configured to activate the actuated structure to maintain the display device in the field of view of the surgeon performing the ophthalmic surgery by:
receive one or more inputs including at least one of:
one or more first camera images received from one or more first cameras mounted to the imaging device;
one or more second camera images received from one or more second cameras mounted to the display device;
one or more third camera images received from one or more second cameras mounted in an operating room including the imaging device an the display device;
first kinematic data for the actuated structure; or
second kinematic data for a support for the imaging device; and
select a position and orientation for the display device based on the one or more inputs; and
activate the actuated structure to move the display device to the position and orientation.

11. A system comprising:
a display device;
an actuated structure mounted to the display device and configured to move the display device in three-dimensional space;
an imaging device configured to capture surgical images of a patient's eye during an ophthalmic surgery and display the surgical images on the display device;
a camera mounted to the imaging device; and
a controller configured to:
receive camera images from the camera;
evaluate representations of the display device in the camera images; and
activate the actuated structure to maintain the display device unobstructed in a field of view of a surgeon performing the ophthalmic surgery based on the representations of the display device.

12. The system of claim 11, wherein the imaging device is an ophthalmic microscope.

13. The system of claim 11, wherein the imaging device is a stereoscopic ophthalmic microscope.

14. The system of claim 13, wherein the display device is a three-dimensional display device and the controller is configured to activate the actuated structure to maintain an orientation and distance of the display device relative to the surgeon to enable three-dimensional perception by the surgeon.

15. The system of claim 11, wherein the controller is further configured to activate the actuated structure to maintain visibility of the display device to the surgeon performing the ophthalmic surgery by:
receiving a surgeon profile;
selecting a position and orientation of the display device according to the surgeon profile; and
activating the actuated structure to achieve the position and orientation.

16. The system of claim 11, wherein the controller is configured to activate the actuated structure to maintain the display device in the field of view of the surgeon performing the ophthalmic surgery by:
receiving a treatment plan for the ophthalmic surgery;
selecting a position and orientation of the display device according to the treatment plan; and
activating the actuated structure to achieve the position and orientation.

17. The system of claim 16, wherein the treatment plan specifies a surgeon position selected from superior, left temporal, and right temporal; and
wherein the controller is further configured to select the position and orientation of the display device such that the display device is visible from the surgeon position in the treatment plan.

18. The system of claim 17, wherein the controller is further configured to select a height of the display device according to a surgeon profile of the surgeon.

19. The system of claim 11, wherein the actuated structure is a robotic arm.

20. The system of claim 11, wherein the actuated structure is a robotic arm having at least 5 degrees of freedom.
